(19) United States
(12) Patent Application Publication
(10) Pub. No.: US 2016/0115440 A1
(43) Pub. Date: Apr. 28, 2016

(54) Microwave enhancement of Thermophiles

(71) Applicant: The Government of the United States of America, as represented by the Secretary of the Navy, Washington, DC (US)

(72) Inventors: Justin C. Biffinger, Woodbridge, VA (US); Lisa A. Fitzgerald, Alexandria, VA (US); Miah J. Davis, Columbia, SC (US); Allison L. Cockrell, Alexandria, VA (US); Kathleen D. Cusick, Alexandria, VA (US)

(21) Appl. No.: 14/918,240

(22) Filed: Oct. 20, 2015

Related U.S. Application Data
(60) Provisional application No. 62/067,545, filed on Oct. 23, 2014.

(51) Int. Cl. C12N 1/20

(52) U.S. Cl. CPC C12N 1/20

(57) ABSTRACT

Microwave radiation was found to increase bacterial growth and cell size compared to conventional heating over a 30 hour time period. One method of growing thermophilic organisms involves heating a growth media comprising a thermophilic organism with microwave radiation to a temperature between 50° C. and 75° C.

### CROSS-REFERENCE TO RELATED APPLICATIONS

This Application claims the benefit of U.S. Provisional Application 62/067,545 filed on Oct. 23, 2014, the entirety of which is incorporated herein by reference.

### BACKGROUND

Thermophilic organisms are those that are capable of surviving and thriving at temperatures greater than 50° C. (see ref. 13). Thermophiles differ from mesophiles based on a variety of features such as genomic guanine-cytosine levels in the coding regions of distinctive genes regardless of their phylogeny, salinity, oxygen availability, or isolation location (see ref. 14). Thermus sp. is one of the most widely encountered thermophiles and is known to produce extracellular proteases, cellulases, and other hydrolases (see ref. 15).

A need exists for techniques for growing thermophilic organisms with microwave (“MW”) radiation.

### BRIEF SUMMARY

In one embodiment, a method of growing thermophilic organisms involves heating a growth media comprising a thermophilic organism with microwave radiation to a temperature between 50° C. and 75° C.

In further embodiments, the growing may take place for a period of one or more hours, or one or more days; and/or until a suitable amount of growth has occurred, for example as measured by an appropriate increase in OD₆₀₀ₙₘ, for example a doubling of OD₆₀₀ₙₘ.

### BRIEF DESCRIPTION OF THE DRAWINGS

FIG. 1 shows growth of Thermus sp. (ATCC® 700910™) at 65° C. using either convective or MW (300W) heating.

FIGS. 2A through 2D show results of viability testing using T. scotoductus SA-01 cells (A) initially and after exposure for 2 h to (B) 300W, (C) 600W, and (D) 1200W MW power (50% duty cycle) maintained at 65° C.

FIGS. 3A through 3F are images of T. scotoductus cells grown in a conventional oven or commercial MW digestor. Scanning electron microscope (SEM) images of cells grown for 24 h under oven (A and B) or MW (D and E) conditions. Images were obtained using a Zeiss scanning electron microscope with the following parameters: extra high tension (EHT), 10.00 kV; working distance (WD), 4.3 mm; magnification, ×1,000 (A and D) and ×3,500 (B and E). Transmission electron microscope (TEM) images of cells grown under oven (C) or MW (F) conditions for 24 h. Images were obtained using a Zeiss EM109 transmission electron microscope operated at 80 kV and at a magnification of ×20,000.

FIG. 4 shows turbidity data from T. scotoductus SA-01 cells (1% inoculum v/v) exposed to mixed heating modes (microwave heating and convective heating) compared to cells only exposed to convective heating over 35 h.

FIG. 5 shows the percentage of genes (relative to total number of differentially regulated genes) expressed at each time point aggregated as clusters of orthologous groups (COGs) that were up or down regulated by microwave heating compared to conventional heating.

FIG. 6 shows growth curves for MW versus conventional heating (“Oven”) with arrows indicating times where the data from FIG. 5 was obtained.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

Described herein is the use of MW dielectric heating to sustain and grow viable thermophilic bacteria. In particular, MW heating can be used to maintain and grow a bacterial culture at a temperature above ambient temperature, for example a temperature between 45° C. and 75° C., more particularly between 50° C. and 70° C., still more particularly at or about 65° C.

Additional details regarding the practice and results of techniques described herein may be found in the published work of the inventors “A comparison of the physical and biochemical properties of Thermus scotoductus SA-01 cultured with microwave radiation and conventional heating,” Appl. Environ. Microbiol. September 2015 vol. 81 no. 18 6285-6293 including the associated supplementary material.

It is possible to create, culture, and manipulate viable prokaryotic and extend those observations to eukaryotic defense mechanisms using MW wavelength (300 MHz-30 GHz) radiation and heating. For this purpose, suitable organisms include thermophilic organisms or thermally stable mesophile mutants that are able to survive at temperatures greater than 50° C.

Heating within a MW is generated from the rotational relaxation induced by the molecular dipole moment. In most biological applications this is the water dipole, but other dipole moments can be excited with this mode of energy (see ref. 9). This effect is described as dielectric heating due to the electromagnetic nature of wave energy. The frequency of the MW radiation rotates the dipole of a molecule exposed to the radiation resulting in a radiation lag due to heating of the solution (see ref. 10). This mode of heating is significantly different from heat transfer through convection since convective heating involves a clear heating gradient. As with MW heating, convective heating can have deleterious effects on living cells. Convective heating also leads to uneven heating of liquid cultures, since the cells closer to the heat source experience significantly more heat energy over time than cells at a greater distance. The convection heating gradient transfer is not applicable to dielectric heating, which is a more even mode of heating and not typically experienced by living organisms. MW effects on isolated enzymes and mesophilic organisms have also been reported for sterilization of materials (see refs. 11, 12).

Low energy MW radiation is typically used in data transmission while higher energy-focused MWs can be used to heat liquids and solids through dielectric heating. Both types can contribute to thermal and non-thermal bioeffects. The existence of a non-thermal effect is an active debate amongst scientists in the field (see refs. 25 and 26). It has been difficult to study non-thermal effects on organisms because MW radiation studies to date have been conducted using mesophiles that cannot tolerate the unavoidable dielectric heating of the medium or organism. Thus, general studies of the non-thermal effects of MW radiation can be achieved through the use of thermophiles, in which thermal effects are removed due to their survival at elevated temperatures.

It is not believed to have been previously shown that thermophiles might survive and grow when elevated temperatures (such as those greater than 40° C.) are generated by MW radiation. In general, living organisms have adapted for experiencing heat transfer from convection through solid, liquid, or gas. Being heated via electromagnetic radiation is similar to heating the entire organism simultaneously. Most of the strategies for improving enzyme stability in thermophiles are in vitro using site-directed mutagenesis or directed evolution (see ref. 27). Thus microwave radiation might mutate the thermophile as a results of this radiation leading to a potential methodology to mutate thermophilic organisms in general. Understanding how a thermophilic microbe survives MW radiation has a wide range of defense and industrial applications as the enzymes from these microbes would be expected to be stable at elevated temperatures and in harsh environments (see ref. 15).

This technique can demonstrate how a living organism interacts with MW radiation and can allow for manipulation of organsims grown under unconventional methods (i.e., MW radiation). The applicable microbes can be screened based on their growth characteristics, membrane integrity, protein/fatty acid distributions, gene transcription, and/or protein expression levels during MW radiation exposure and subsequent heating.

MW heating surprisingly and dramatically increased the growth rate so that the overall amount of biomass in the culture was >80% compared to convection heating at the same temperature (65° C.). This is counter to present day knowledge, as MW radiation is commonly used to destroy microbes as in the sterilization of meats and fruits (see ref. 6) and treatment of dairy products (see ref. 18), rather than growing them. In addition, published work on microorganisms and MWs largely relates to processing biomass and eradicating microorganisms using MW radiation, and not the intentional culturing of thermophiles under sustained MW radiation.

Compared to conventional heating, MW devices reduce heat transfer resistances, since the heating occurs at a molecular level and not on the chamber wall of vessels that absorb microwave radiation readily. Therefore, MW processing results in higher heating efficiencies and better heating rates for a given power. Manipulating and growing thermally stable organisms in MW radiation may result in new classes of proteins, enzymes, and organisms suitable for industrial commodity chemicals or fuels (see ref.16). In addition to the ability to survive MW radiation, the elevated temperatures can be advantageous for accelerated reaction rates and product distributions (see refs. 5 and 7). Understanding living organisms grown in a MW could create living templates for nanoparticle formation, which could lead to in-situ nanoparticle catalysts on living microorganism supports (see ref. 24).

An early report of using MWs for a biological application was in 1924 for reducing tumor formation in plants (see ref. 1), and a design of a MW oven was patented in 1977 (see ref. 2). MW technology has also been used in a wide variety organic and inorganic transformations to improve yields and rates of reactions (see ref. 3). More recently, MWs have been used in biological reactions with isolated proteins or enzymes (see refs. 4-7). The MW region of the electromagnetic spectrum (300 MHz-30 GHz) is between radio and infrared frequencies. With most RADAR and telecommunication transmissions existing between 29 GHz-1 GHz, commercial MW heating apparatuses typically operate at 2.45 GHz. MW frequencies >30 GHz are extra high frequencies and generally reserved for satellite-to-earth communication. MWs interact with living and non-living organisms in various ways that might be considered adverse (cell lysis or triggering apoptosis) and/or beneficial with thermophiles (heating or growth) (see ref. 8).

Particular embodiments may include growing thermophiles in an enclosed chamber where the interior is subject to MW radiation, or in a partially enclosed area, or apart from an enclosure wherein MWs are beamed at the thermophiles from one or more sources.

Growth and studies of thermophilic organisms within a MW have applications for understanding and manipulating an organism's resistance to focused MW radiation. This is due to the survivability of the thermo-tolerant cells and mutants to dielectric heating which is not known to occur in mesophiles when cultured in a MW. In view of the present discovery that a thermophile can actively grow in focused MW radiation, resulting data on cellular metabolism, gene transcription, and protein activity could provide insight as to how MW radiation interacts with living prokaryotic and eukaryotic organisms. Such findings might then be used in the development of new methods to either enhance or destroy pathogenic thermophiles.

It is expected that growing and studying living thermophilic organisms in MW-irradiated environments can lead to better understandings of how MW radiation interacts with various forms of life (including human), and allow development of new technologies (such as fuel and waste remediation strategies) using thermophilic organisms exposed to MWs. Thermophiles actively growing in a MW allows for the first unambiguous study of non-thermal MW effects resulting in a more rapid determination of the impact (gene transcription, protein expression, and secretion) of MW radiation on living organisms. How these thermophiles respond to the MW treatment on a physiological level is extremely important to understanding how enlisted military might be responding to MW radiation exposure with reference to gene transcription/translation pathways. Secondly, proteins in thermophiles, like Thermus sp., can have activity in extreme environments that the same class of proteins isolated from mesophilic organisms will not. This can lead to higher activities of enzymes that could be used for industrially relevant processes (see refs. 10 and 19).

Culturing a thermophile in a commercial MW also allows for longer culturing periods (>30 min) and unprecedented exposure times to MW radiation. This is an advantage for understanding long-term effects of MW radiation on living microorganisms.

Changes to proteins as a result of being grown using MW radiation could lead to a new class of MW-tolerant enzymes and proteins: engineered proteins could be screened by producing them under MW radiation and then testing their function and/or structural attributes.

An additional application is the reduction of metals to nanoparticles by using living, metal-reducing thermophiles under MW radiation—these might be used in both industrial and defense applications or the remediation of wastewater or reclamation of metals from wastewater (see refs. 20-22). Each of these processes could be enhanced if stimulating the organism with MW radiation results in defined molecular changes. The generation of reactive molecules (for example H₂ (see refs. 17 and 23), CO₂, CH₄, ethylene, butanol) catalyzed by the living organisms exposed to MWs could power shipboard electricity (nuclear, no carbon release) or be installed into the electric grid (controlled and limited carbon release) with less power used overall due to the efficiency of MW heating of the living cells.

The discovery of survival of thermophiles in MWs, as disclosed herein, opens numerous avenues for further research and industrial applications. The data collected from thermophiles cultured in MW radiation can lead to the resolution of potential hazards with regards to their survival in food for active military and the domestic population. Understanding how an organism can survive in a MW will lead to potential mechanisms and the identification of how biological threats and agents could survive standard radiation protocols as well.

Methods and Results

An exemplary thermophile, T scotoductus, SA-01 (ATCC 700910), was cultured and manipulated. This organism was previously isolated from groundwater from a gold mine in South Africa and is typically cultured at 65° C. General culture characteristics using only convective heating were reported by Kieft and coworkers in 1999 (see ref. 28). Cell densities were typically between 6×10⁷ and 9×10⁷ CFU/mL and cells typically grew with a lag phase around 25 hours.

Growth of T. scotoductus within a MW Oven Compared to Convective Heating

A culture of T. scotoductus SA-01 was inoculated using a frozen stock made from cells originally purchased from the American Type Culture Collection (ATCC) as strain number 700910. The culture was grown aerobically with shaking at 65° C. in 10 mL of 461 Castenholz TYE medium (formulation modified from the ATCC recipe by using CaCl₂ instead of CaSO₄). Once the culture cell density (determined by visible spectroscopy at 600 nm) was greater than 1.0, 2 mL (10% inoculation v/v) of that culture was used to inoculate 60 mL of 461 Castenholz TYE medium in a Teflon® decomposition reactor (100 mL total volume). Parallel experiments were performed using identical inoculation size to test growth under standard conditions (convective oven set to 65° C.) and heating in a MW digestion device (MARS5™ manufactured by CEM Corporation). For the MW studies, an IR fiber optic temperature probe was placed in a control vessel containing 60 mL of the same medium used for the experiments. The power output from the MW was 300W (50% duty cycle) for a 3 min ramp to 65° C. then the temperature was held at 65° C. for 120 min. The frequency 2450 MHz has a length of 12.2 cm, which has an appropriate penetration depth (the distance a MW can travel into a standard sample) for use with small samples. Aliquots were removed periodically to determine the cell density of the solution under each condition by visible spectroscopy at 600 nm.

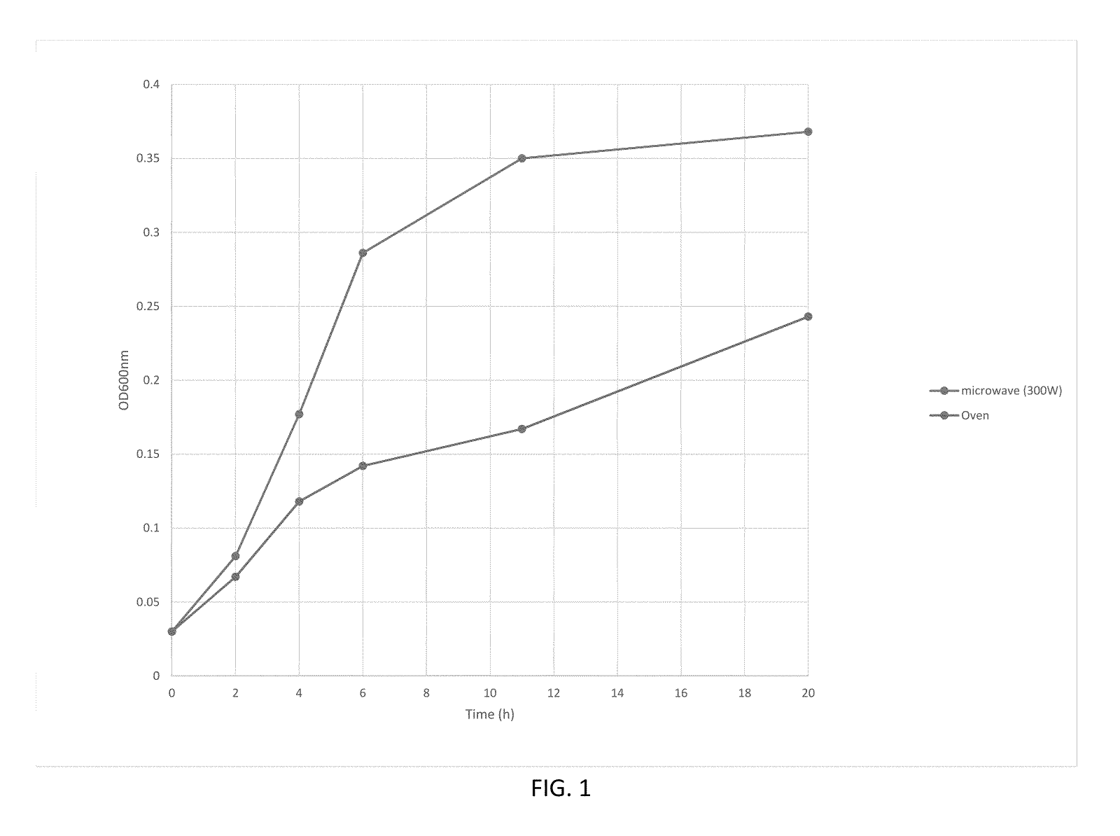

FIG. 1 shows growth of T. scotoductus SA-01 at 65° C. using either convective or MW (300W) heating. These data show that the Thermus sp. actively grew within a MW chamber at 65° C. to a higher density than cells grown with convective heating (˜0.35 vs. ˜0.25, respectively). The temperature was maintained in the MW because the heating program on the Mars5 was designed to modulate the duty cycle from the MW to maintain a temperature in the control vessel. Since the liquids and vessels in both the oven and MW systems were identical there were no differences in the heating characteristics of the medium. This was also confirmed by independent temperature measurements of the liquids after heating in the MW or convective oven.

Cell Viability after Heating with MW Power

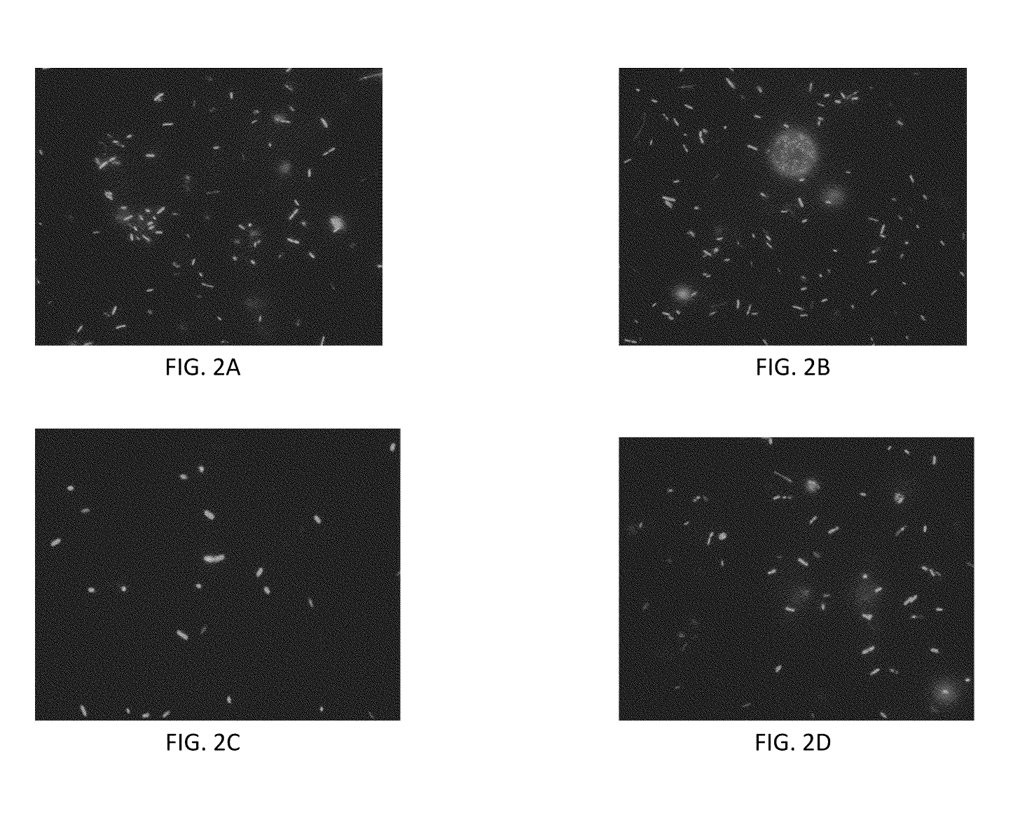

To grow a culture of T. scotoductus SA-01 in TYE, it was first inoculated using a frozen stock of T. scotoductus cells made from lyophilized cultures purchased from www.ATCC.org (ATCC 700910). The culture was grown aerobically with shaking at 65° C. Once the culture cell density (determined by visible spectroscopy at 600 nm) was greater than 1.0, 2 mL (10% inoculation v/v) of that culture were used to inoculate 60 mL of 461 Castenholz TYE medium in a Teflon® decomposition reactor (100 mL total volume). Parallel experiments were performed using identical inoculation size to test growth under standard conditions (convective oven set to 65° C.) and heating in a MW digestion device MARS5^{TM}, manufactured by CEM Corporation). Each vessel was placed in the MW chamber separately and different power outputs (300W, 600W, and 1200W) were chosen for the heating of the cells within the MW. The frequency 2450 MHz has a wavelength of 12.2 cm, which is an appropriate penetration depth (the distance a MW can travel into a standard sample) with these samples. The resulting Live/Dead viability assay is shown in FIG. 2. Green cells are viable cells while dead cells have compromised cell membranes and appear red. The cells in FIG. 2A were maintained in a convective oven during the same time period that cells were exposed to MW heating. This experiment shows that the cells in the MW at a variety of power output levels remained viable and that MWs were not adversely affecting the viability of the cells. These results suggest that it possible to use MW radiation to culture and manipulate thermostable microorganisms.

FIG. 2 shows live/dead viability stain of T. scotoductus SA-01 cells (A) initially and exposed for 2 h to (B) 300W, (C) 600W, and (D) 1200W MW power (50% duty cycle) maintained at 65° C.

Morphological Changes

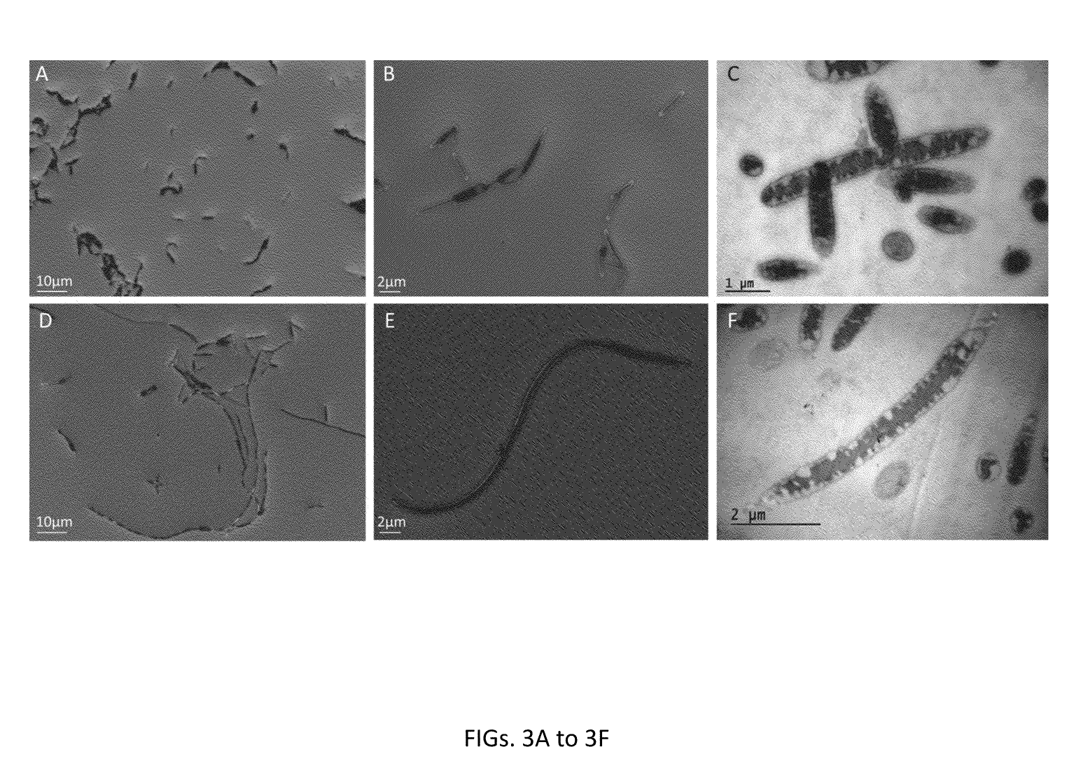

SEM images showed that the oven-grown cells were ˜2 μm long and well dispersed (FIGS. 3A and B). MW-grown cultures, however, contained a greater distribution of elongated cells and contained aggregated cells and “chains” of cells (attached at the ends) that were not observed in oven-grown cultures (FIGS. 3D and E). MW samples contained cells as short as 2 μm, with chains of cells reaching over 100 μm in length. Both TEM and atomic force microscopy (AFM) topography imaging showed that oven-grown cells were smaller than MW-grown cells (FIG. 3C and F show TEM images—the AFM data is not shown). Comparisons of cell morphology indicated that the MW-grown cells were longer and narrower than the oven-grown cells Imaging results confirmed general trends found in dynamic light scattering (DLS) experiments and demonstrated that 24 h of MW exposure led to aggregation and elongation of T. scotoductus cell cultures.

Additional data showing these morphological differences can be seen in the above-mentioned document Appl. Environ. Microbiol. September 2015 vol. 81 no. 18 6285-6293 and its supplemental material.

Effect of MWs on Cell Viability

The impact of MW power on cell viability was also examined. Fluorescent microscopy images of T. scotoductus SA-01 (purchased from www.ATCC.org) cells stained with BACLIGHT Live/Dead Viability stain were taken after exposure to MW heating at 300W, 600W, and 1200W for 1 hr at a 50% duty cycle. The MW was a CEM Mars5 synthetic MW (CEM Corp.) with Teflon vessels. The fiber optic temperature probe was placed in a vessel containing 461 Castenholz TYE growth medium and was used as the internal standard for temperature. As the MW reached the set temperature, the MW regulated the temperature by decreasing the duty cycle to approximately 10% from the initial 50%. The temperature regulation from the CEM Mars5 synthetic MW allowed for the sample to maintain a temperature by exposing the cultures to different levels of power from the MW. The MW-grown cells were compared to cells exposed to convective heating at 65° C. Live/Dead staining confirmed the cells maintained viability when exposed to MWs and convective heating.

Growth under MW Radiation with 1% Inoculum

MW growth was studied in TYE broth with 1% inoculum. 60 mL quantities of TYE broth (ATCC formulation) were added to four 100 mL Teflon vessels. 200 μL of a 0.779 OD₆₀₀ₙₘ. T. scotoductus (SA-01, purchased from www.ATCC.org) culture grown with shaking at 65° C. in TYE was added to each vessel at room temperature. The initial OD was recorded and one vessel was placed in the CEM Mars5 synthetic MW with Teflon vessels as described above. The fiber optic temperature probe was placed in a vessel containing the growth medium and was used as the internal standard for temperature. The power for this experiment was 300W (50% duty cycle) with a 3 min heating time followed with 120 min of incubation with MW exposure. Aliquots were removed every 2 h and optical densities (OD) were recorded by UV-Visible spectroscopy (UV-vis) at 600 nm.

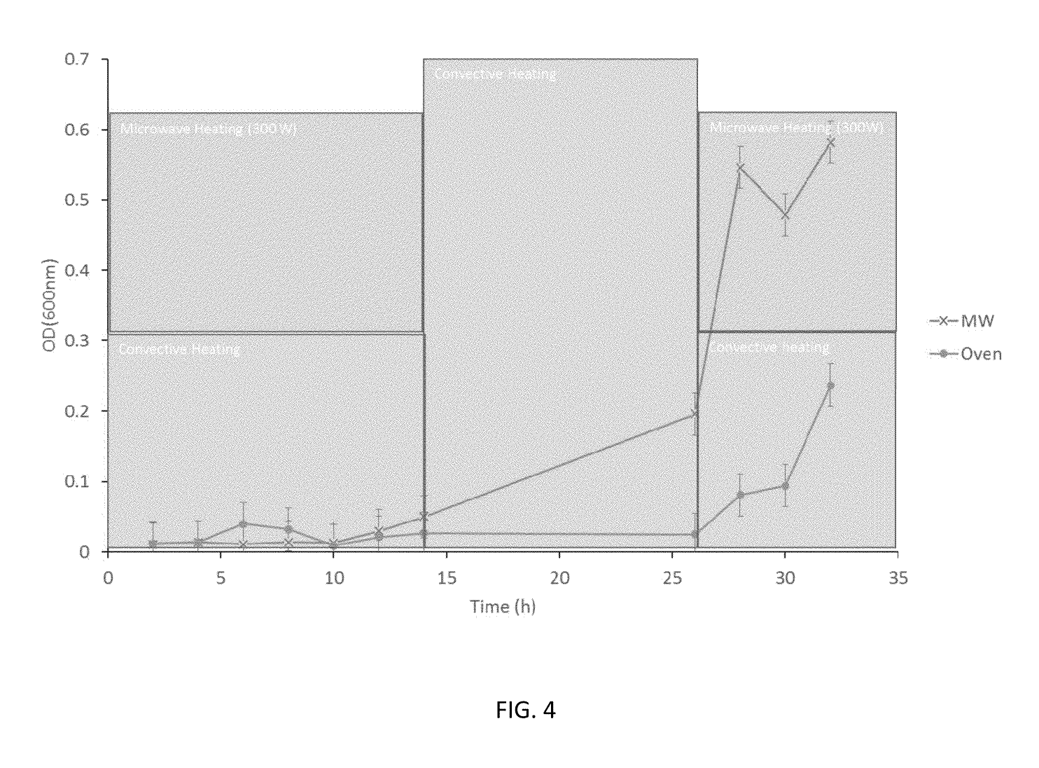

FIG. 4 shows turbidity data from T. scotoductus SA-01 growth (1% inoculum v/v) exposed to mixed heating modes (MW heating and convective heating) compared to cells only exposed to convective heating.

Comparative Growth with 1%, 5%, or 10% Inoculum

A comparison was made between MW and convective growth in TYE with 1%, 5%, or 10% inoculum. 40 mL quantities of TYE broth (formulation from ATCC) were added to several 100 mL Teflon vessels. 400 μL, 2 mL, or 4 mL of a 1.045 OD6₀₀ₙₘ T. scotoductus (SA-01, purchased from ATCC) culture grown with shaking at 65° C. in TYE was added to each vessel at room temperature. The initial OD was recorded and one vessel was placed in the CEM Mars5 synthetic MW (CEM Corporation) with Teflon vessels as above and the other corresponding vessel was placed in a 65° C. incubator. The fiber optic temperature probe was placed in a vessel containing the growth medium and was used as the internal standard for temperature. The power for this experiment was 300W (50% duty cycle) with a 3 min heating cycle followed by 120 min of MW exposure. Aliquots were removed every 2 h and optical densities were recorded by UV-vis (Abs=600 nm).

Transcriptional Changes

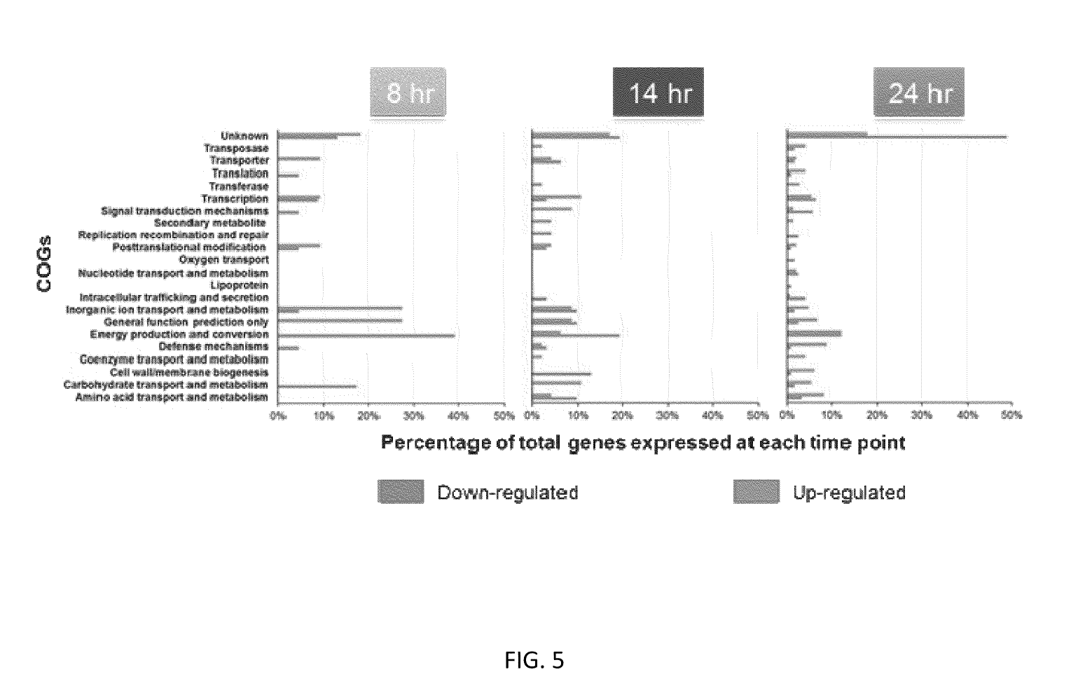

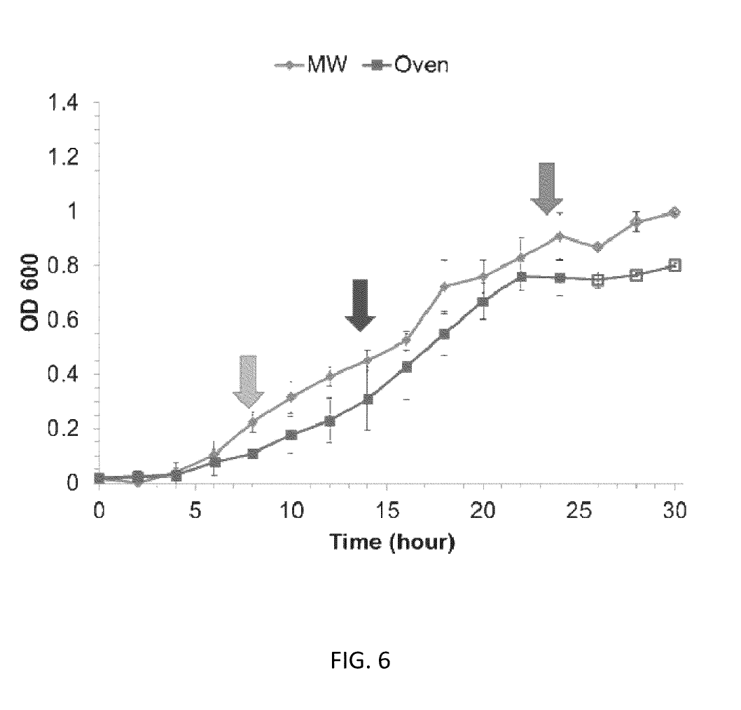

FIG. 5 shows clusters of orthologous groups (COGs) that were up or down regulated by MW heating compared to conventional heating. The percentage of total gene expressed were compared to the total amount of genes differentially expressed at each time point. Data was obtained from RNAseq transcriptional data at three time points during the growth of T. scotoductus, with the three points indicated by arrows in FIG. 6. These data suggest that MW radiation induces cell wall changes from a stress response induced by the MW heating at the last time point alone.

Applications

Thermophiles, including genetically engineered thermophiles, might be used to generate proteins including industrially important enzymes. Such thermo-tolerant enzymes can have significant impact on the production of commodity chemicals and degradation of lignocellulosic materials. The end products of these processes are fuels, additives, polymers, and specialty chemicals.

The cultivation of thermophiles at high temperatures has potential economic advantages since the risk for contamination and fluid viscosity is lower which can lead to large economic advantages for engineering a reactor necessary for industrial process (see refs. 15-17). One of the major drawbacks to using thermophiles with convective heating are the typically low cell densities generated which has led to research into costly growth media formulations and conditions. However, the use of MW radiation as presented herein appears to stimulate larger quantities of biomass, when compared to conventional heating, which is a huge roadblock to using the microbes as whole cell reactors. One practical example of a possible benefit from the development of methodologies for and isolation of enzymes from thermophilic organisms might be enzymes that would only respond to MW radiation and heating. This could then be used as a mechanism to either stimulate or deactivate enzymatic processes which could be used for a particular process.

The techniques described herein may be used to study effects of MW radiation on living cells. It could also be possible to apply them for bioremediation of wastewater and halogenated compounds to decrease environmental hazards. Thermophiles grown under MW radiation could be used in nanoparticle formation within a MW as a method for creating commodity chemicals and fuels, or similarly MWs may be used for stimulation of precious metal reduction on the surface of a thermophilic organism.

Furthermore, it may be possible to elucidate mechanisms to deactivate pathogen thermophilic organisms from surfaces and food and liquid materials; and/or to cultivate or destroy MW tolerant thermophilic mutants and proteins. It may even be possible to use these techniques for long duration regenerative fuel production for interstellar missions.

This technique capitalizes on the concept that growing thermophilic microorganisms within a MW can change the overall morphology of the organism. However, due to the mechanism of heating, it does impact cellular division and thus proteins, cofactors, or gene transcription levels. These are fundamental changes to the cells that can be distinguished from thermal and non-thermal stimuli. Microorganisms which are able to survive MW heating can thrive under conditions leading to potential mechanisms that will improve cellular densities and increase protein, enzyme, or fatty acid yields that could prove useful in fuel or any commodity chemical process. This advance may relate to the need to find and utilize renewable fuel streams separate from primary food sources (corn, soy beans, etc.) and the applicability of thermophilic organisms for cellulose degradation could lead to an advancement of second-generation biofuels. This discovery could also lead to manipulating organisms to survive MW sterilization which has a significant downstream impact on food safety and homeland security issues.

It is expected that nearly any thermophilic organism will be able to survive MW heating and thus be used for the techniques described herein. Exemplary organisms include, but are not limited to, moderate thermophiles such as members of the genera Geobacillus, Thermoanaerobacter and Clostridium and hyperthermophiles including the Archaeal species of the genera Sulfolobus, Pyrodictium, Pyrolobus, Pyrobaculum, Thermoproteus, Thermococcus, Pyrococcus as well as other genera. Other suitable examples include bacteria of the Thermales order including Thermus sp. such as Thermus scotoductus.

In general, this is a novel method to generate MW enhanced organisms and MW tolerant proteins/enzymes for use in models for heat-tolerant microorganisms, new classes of native proteins and enzymes for fuels, lubricants or additives, and other industrial processes that require catalysts at elevated temperatures. Applications may include the enzymatic degradation of abundant materials, often waste products, such including lignocellulose and chitin, to produce fuel as described in ref. 16, incorporated herein by reference for teachings relating to the production of biofuel.

Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Mishra, T., Kushwah, P., Dholiya, K. & Kothari, V. Effect of Low Power Radiation on Microorganisms and other Life Forms. Advances in Microwave and Wireless Technology 1, 4-11 (2013).
2. U.S. design Pat. No. D243994, granted Apr. 12, 1977.
3. Lidstrom, P., Tierney, J., Wathey, B. & Westman, J. Microwave assisted organic synthesis: a review. Tetrahedron 57, 9225-9283 (2001).
4. Sontakke, J. B. & Yadav, G. D. Microwave Assisted Synthesis of Ethyl 2-(4-aminophenyl) Acetate Using Novozyme 435. Current Catalysis 3, 27-34 (2014).
5. Devendran, S. & Yadav, G. D. Microwave assisted enzymatic kinetic resolution of (±)-1-Phenyl-2-propyn-1-ol in nonaqueous media. BioMed Research International, 482678/482671-482678/482610 (2014).
6. Benlloch-Tinoco, M., Igual, M., Rodrigo, D. & Martinez-Navarrete, N. Comparison of microwaves and conventional thermal treatment on enzymes activity and antioxidant capacity of kiwifruit puree. Innovative Food Science & Emerging Technologies 19, 166-172 (2013).
7. Yadav, G. D. & Thorat, P. A. Microwave assisted lipase catalyzed synthesis of isoamyl myristate in solvent-free system. Journal of Molecular Catalysis B: Enzymatic 83, 16-22 (2012).
8. Szmigielski, S. Reaction of the immune system to low-level RF/MW exposures. Science of The Total Environment 454-455, 393-400 (2013).
9. Tyagi, V. K. & Lo, S.-L. Microwave irradiation: A sustainable way for sludge treatment and resource recovery. Renewable & Sustainable Energy Reviews 18, 288-305 (2013).
10. Chandrasekaran, S., Ramanathan, S. & Basak, T. Microwave material processing-a review. AIChE Journal 58, 330-363 (2012).
11. Singh, R. & Singh, D. Sterilization of bone allografts by microwave and gamma radiation. International Journal of Radiation Biology 88, 661-666 (2012).
12. Wu, Y. & Yao, M. Inactivation of bacteria and fungus aerosols using microwave irradiation. Journal of Aerosol Science 41, 682-693 (2010).
13. Cava, F., Hidalgo, A. & Berenguer, J. Thermus thermophilus as biological model. Extremophiles 13, 213-231 (2009).
14. Zheng, H. & Wu, H. Gene-centric association analysis for the correlation between the Guanine-Cytosine content levels and temperature range conditions of prokaryotic species. BMC Bioinformatics 11, S7 (2010).
15. Turner, P., Mamo, G. & Karlsson, E. N. Potential and utilization of thermophiles and thermostable enzymes in biorefining. Microbial Cell Factories 6, No pp given (2007).
16. Taylor, M. P., Bauer, R., Mackay, S., Tuffin, M. & Cowan, D. A. Extremophiles and their application to biofuel research. Extremophiles, 233-264, 265 (2013).
17. Kivisto, A. T., Ciranna, A., Santala, V. P. & Karp, M. T. Extremophilic microbes in biohydrogen production. Current Biotechnology (Sharjah, United Arab Emirates) 2, 345-359 (2013).
18. Geczi, G., Horvath, M., Kaszab, T. & Alemany Gonzalo, G. No major differences found between the effects of microwave-based and conventional heat treatment methods on two different liquid foods. PLoS One 8, e53720 (2013).
19. Shamis, Y., Croft, R., Taube, A., Crawford, R. J. & Ivanova, E. P. Review of the specific effects of microwave radiation on bacterial cells. Applied Microbiology and Biotechnology 96, 319-325 (2012).
20. Pattrick, R. A. D. et al. Extracellular bacterial production of doped magnetite nanoparticles. Nanoscience (Cambridge, United Kingdom) 1, 102-115 (2013).
21. Varshney, R., Bhadauria, S. & Gaur, M. S. A review: biological synthesis of silver and copper nanoparticles. Nano Biomedicine and Engineering 4, 99-106 (2012).
22. Omole, M. A., K'Owino, I. & Sadik, O. A. Nanostructured materials for improving water quality: potentials and risks. Nanotechnology Applications for Clean Water, 233-247 (2009).
23. Faloye, F. D., Gueguim Kana, E. B. & Schmidt, S. Optimization of biohydrogen inoculum development via a hybrid pH and microwave treatment technique-Semi pilot scale production assessment. International Journal of Hydrogen Energy 39, 5607-5616 (2014).
24. Parsons, J. G., Peralta-Videa, J. R., Dokken, K. M. & Gardea-Torresdey, J. L. Biological and biomaterials-assisted synthesis of precious metal nanoparticles. Nanomaterials for the Life Sciences 1, 461-491 (2009).
25. Porcelli, M. et al. Non-thermal effects of microwaves on proteins: thermophilic enzymes as model system. FEBS Letters 402, 102-106 (1997).
26. Fortune, J. A., Wu, B.-I. & Klibanov, A. M. Radio frequency radiation causes no nonthermal damage in enzymes and living cells. Biotechnology Progress 26, 1772-1776 (2010).
27. Short, et al., U.S. Pat. No. 6,713,281.
28. Kieft, T. L. et al. Dissimilatory reduction of Fe(III) and other electron acceptors by a Thermus isolate. Applied and Environmental Microbiology 65, 1214-1221 (1999).

What is claimed is:

1. A method of growing a thermophilic organism, the method comprising:
heating a growth media comprising a thermophilic organism with microwave radiation to a temperature between 50° C. and 75° C., thereby causing the organism to multiply.

2. The method of claim 1, wherein the heating occurs for a period of time sufficient to cause a doubling of OD₆₀₀ₙₘ.

3. The method of claim 1, wherein the thermophilic organism is Thermus sp.

4. The method of claim 3, wherein the thermophilic organism is Thermus scotoductus.

5. The method of claim 1, wherein differential gene transcription occurs in the absence of microwave radiation.

6. The method of claim 1, wherein the organism produces an enzyme suitable for fuel production.
